(19) United States
(12) Patent Application Publication
(10) Pub. No.: US 2007/0161679 A1
(43) Pub. Date: Jul. 12, 2007

(54) Method and compositions for the intravenous administration of compounds related to proton pump inhibitors

(75) Inventor: Patrick M. Hughes, Aliso Viegjo, CA (US)

Correspondence Address: ALLERGAN, INC., 2525 DUPONT DRIVE, T2-7H, IRVINE, CA, 92612-1599, US

(73) Assignee: Allergan, Inc., Irvine, CA (US)

(21) Appl. No.: 10/597,804

(22) PCT Filed: Jan. 14, 2005
(86) PCT No.: PCT/US05/01462

§ 371 Date: Aug. 8, 2006

Related U.S. Application Data
(60) Provisional application No. 60/545,809, filed on Feb. 18, 2004.

(51) Int. Cl. A61K 31/4439

(52) U.S. Cl. 514/338

(57) ABSTRACT

Disclosed herein are liquid compositions comprising a therapeutically effective concentration of a prodrug of a sulfonyl-containing proton pump inhibitor, wherein said compositions have a pH of from 3 to 7.3. Solid compositions related thereto are also disclosed. Methods of delivering a proton pump related thereto are also disclosed. Compositions comprising a second therapeutically active agent are also disclosed herein.

### BACKGROUND OF THE INVENTION

1. Field of the Invention

The present invention is directed to compositions and methods comprising compounds related to proton pump inhibitors, which are useful as inhibitors of gastric acid secretion.

2. Description of the Related Art

Benzimidazole derivatives intended for inhibiting gastric acid secretion are disclosed in U.S. Pat. Nos. 4,045,563; 4,255,431; 4,628,098; 4,686,230; 4,758,579; 4,965,269; 5,021,433; 5,430,042 and 5,708,017. Generally speaking, the benzimidazole-type inhibitors of gastric acid secretion are believed to work by undergoing a rearrangement to form a thiophilic species which then covalently binds to gastric H,K-ATPase, the enzyme involved in the final step of proton production in the parietal cells, and thereby inhibits the enzyme. Compounds which inhibit the gastric H,K-ATPase enzyme are generally known in the field as “proton pump inhibitors” (PPI).

Some of the benzimidazole compounds capable of inhibiting the gastric H,K-ATPase enzyme have found substantial use as drugs in human medicine and are known under such names as LANSOPRAZOLE (U.S. Pat. No. 4,628,098), OMEPRAZOLE (U.S. Pat. Nos. 4,255,431 and 5,693,818), ESOMEPRAZOLE (U.S. Pat. No. 6,369,085) PANTOPRAZOLE (U.S. Pat. No. 4,758,579), and RABEPRAZOLE (U.S. Pat. No. 5,045,552). Some of the diseases treated by proton pump inhibitors and specifically by the five above-mentioned drugs include peptic ulcer, heartburn, reflux esophagitis, erosive esophagitis, non-ulcer dyspepsia, infection by Helicobacter pylori, alrynitis and asthma.

Although proton pump inhibitors have been found to be generally useful for the treatment of acid-related gastrointestinal disorders, intravenous administration of proton pump inhibitors has been problematic due to their instability in aqueous solutions, even at near neutral and basic pH. For example, the only FDA-approved intravenous proton pump inhibitor therapy is Protonix® I.V., which uses pantoprazole sodium as the active ingredient. Like omeprazole and the other commercial proton pump inhibitors, the rate of pantoprazole degradation increases with decreasing pH, and as a result of this instability, Protonix® I.V. is administered in a reconstituted solution at a pH between 9 and 10. Due to the high pH of the reconstituted formula, slow infusion of the drug over a period of 15 minute is required to minimize irritation at the injection site. Additionally, the reconstituted formula may not be stored for more than 12 hours at room temperature. As a result, any improvement that allows the administration of the proton pump inhibitor to be accomplished at a more neutral pH would be a significant contribution to the art.

As further pertinent background to the present invention, applicants note the concept of prodrugs which is well known in the art. Generally speaking, prodrugs are derivatives of per se drugs, which after administration undergo conversion to the physiologically active species. The conversion may be spontaneous, such as hydrolysis in the physiological environment, or may be enzyme catalyzed. From among the voluminous scientific literature devoted to prodrugs in general, the foregoing examples are cited: Design of Prodrugs (Bundgaard H. ed.) 1985 Elsevier Science Publishers B. V. (Biomedical Division), Chapter 1; Design of Prodrugs: Bioreversible derivatives for various functional groups and chemical entities (Hans Bundgaard); Bundgaard et al. Int. J. of Pharmaceutics 22 (1984) 45-56 (Elsevier); Bundgaard et al. Int. J. of Pharmaceutics 29 (1986) 19-28 (Elsevier); Bundgaard et al. J. Med. Chem. 32 (1989) 2503-2507 Chem. Abstracts 93, 137935y (Bundgaard et al.); Chem. Abstracts 95, 138493f (Bundgaard et al.); Chem. Abstracts 95, 138592n (Bundgaard et al.); Chem. Abstracts 110, 57664p (Alminger et al.); Chem. Abstracts 115, 64029s (Buur et al.); Chem. Abstracts 115, 189582y (Hansen et al.); Chem. Abstracts 117, 14347q (Bundgaard et al.); Chem. Abstracts 117, 55790x (Jensen et al.); and Chem. Abstracts 123, 17593b (Thomsen et al.).

A publication by Sih., et al. (Journal of Medicinal Chemistry, 1991, vol. 34, pp 1049-1062), describes N-acyloxyalkyl, N-alkoxycarbonyl, N-(aminoethyl), and N-alkoxyalkyl derivatives of benzimidazole sulfoxide as prodrugs of proton-pump inhibitors. According to this article these prodrugs exhibited improved chemical stability in the solid state and in aqueous solutions, but had similar activity or less activity than the corresponding parent compounds having a free imidazole N—H group

U.S. Pat. No. 6,093,734 and PCT Publication WO 00109498 (published on Feb. 24, 2000) describe prodrugs of proton pump inhibitors which include a substituted arylsulfonyl moiety attached to one of the benzimidazole nitrogens of proton pump inhibitors having the structure identical with or related to proton pump inhibitor drugs known by the names LANSOPRAZOLE, OMEPRAZOLE, PANTOPRAZOLE and RABEPRAZOLE.

PCT Publication WO 02/30920 describes benzimidazole compounds which are said to have gastric acid secretion inhibitory and anti H. pylon effects. PCT Publication WO 02/00166 describes compounds that are said to be nitric oxide (NO) releasing derivatives of proton pump inhibitors of the benzimidazole structure.

U.S. Patent Application having the title “PRODRUGS OF PROTON PUMP INHIBITORS”, filed Jul. 15, 2003 by applicants Michael E. Garst, George Sachs, and Jai M. Shin, which has not yet been assigned a serial number, discloses prodrugs of the proton pump inhibitor type drugs having an arylsulfonyl group with an acidic functional group attached, which provided improved solubility in physiological fluids and improved cell penetration.

### BRIEF DESCRIPTION OF THE INVENTION

Disclosed herein are compositions comprising a therapeutically effective concentration of an N-phenylsulfonyl prodrug of a proton pump inhibitor comprising a solubilizing moiety, wherein said compositions are aqueous liquids having a pH of from 3 to 7.3.

Other embodiments relate to a solid composition comprising a prodrug of a proton pump inhibitor comprising a sulfonyl moiety and a carboxylic acid or a pharmaceutically acceptable salt thereof, said solid composition having a pH which is greater than 3 and less than or equal to 7 when dissolved in water at a therapeutically effective concentration for intravenous administration of said prodrug.

Also disclosed herein is a method of delivering a proton pump inhibitor to a mammal. This method comprises dissolving in an aqueous solution a therapeutically effective amount of a proton pump inhibitor which is coupled to an ionic functional group or a conjugate acid or base thereof via a sulfonamide linkage. Said aqueous solution is administered parenterally to said mammal; wherein said aqueous solution has a pH which is greater than or equal to 3 and less than 7.

Also disclosed herein is a liquid composition comprising a sulfonamide of a proton pump inhibitor and a second therapeutically active agent, said composition having a pH of from 3 to 8.

Also disclosed herein is a solid composition comprising a sulfonamide of a proton pump inhibitor and a second therapeutically active agent, said composition having a pH of from 3 to 8 when said composition is dissolved in water at a concentration that is therapeutically effective for parenteral administration of the sulfonamide of a proton pump inhibitor.

### DETAILED DESCRIPTION OF THE INVENTION

While not intending to limit the scope of the invention, or be bound in any way by theory, we have discovered that certain compounds disclosed herein have the unexpected property that they are significantly more stable than proton pump inhibitors in aqueous solutions in certain pH ranges, but are rapidly converted to proton pump inhibitors in vivo, such that the proton pump inhibitors are effectively delivered to a mammal. As mentioned previously, the aqueous instability of proton pump inhibitors in certain pH ranges has limited intravenous delivery of these drugs to individuals in need of their gastric secretion inhibition properties. As such, while not intending to limit the scope of the invention in any way, this disclosure is considered to be particularly relevant to improving upon the current methods of intravenous administration of proton pump inhibitors and related compounds to mammals.

Certain embodiments disclosed herein relate to the pH of aqueous compositions disclosed herein. While not intending to limit the scope of the invention in any way, the stability of the compounds disclosed herein in aqueous solutions may confer significantly more flexibility in terms of pH. While not intending to limit the scope of the invention in any way, this flexibility is important because allows the prodrug to be injected in a liquid which has a pH which is more comfortable for the mammal to which the prodrug is administered. Additionally, while not intending to limit the scope of the invention, the prodrugs disclosed herein have significantly greater stability at lower pH, and thus allow significantly greater flexibility in terms of co-administration of other drugs compared to the parent proton pump inhibitors. The table below shows the pH ranges where certain drugs, which are useful to co-administer with proton pump inhibitors or their prodrugs, are most stable. Thus, a composition disclosed herein might be in one or more of the most stable pH ranges for each drug disclosed in the table below. In other compositions the pH is in a range which has a lower limit in accordance with one of the lower limits of one of the most stable pH ranges below, but has an upper limit which is 7.3, 7.0, less than 7, or 5. Alternatively, a composition may have a pH in a range which has an upper limit in accordance with one of the upper limits of one of the most stable pH ranges below, but has a lower limit which is 3, over 3, or 5.

| Drug | Most stable pH range |
|---|---|
| Cefotaxime sodium | 5.5 |
| Intralipi | 5.5 |
| Morphine | 2.5-6.0 |
| Indomethacin | 6.0-7.5 |
| Heparing | 5.5-8.0 |
| Fentanyl | 4.0-7.5 |
| Dopamine | 2.5-4.5 |
| Caffeine | 2.0-3.0 |

Certain compositions comprising the prodrug have a pH which is in a range which provides great flexibility in terms of compatibility with other drugs. One composition has a pH of from 3 to about 8. Another composition has a pH of from 5 to 6. Another composition has a pH of about 5.5. Another composition has a pH of about 6. Another composition has a pH of from 5 to 7. Another composition has a pH of from 3 to 6.

In certain compositions, the prodrug is present without a second drug, and the drug is added to the IV composition just prior to administration. In other compositions, the prodrug and the second drug, or therapeutically active agent, are present in a single composition which is reconstituted or otherwise prepared for IV or another form of parenteral administration.

The term “prodrug” has the meaning previously described herein, and in relation to this disclosure refers to a prodrug of a proton pump inhibitor. The term “proton pump inhibitor” also has the meaning previously described herein.

Certain compounds have been shown to be useful as prodrugs in relation to the embodiments disclosed herein. In certain embodiments, the prodrug comprises a sulfonyl moiety. A “sulfonyl” moiety is defined herein as a moiety comprising an SO₂ group, where a sulfur atom is directly covalently bonded to two oxygen atoms. In other embodiments, the prodrug comprises a phenylsulfonyl moiety. The term “phenylsulfonyl” moiety should be broadly interpreted to mean any moiety where the sulfur of the SO₂ group is directly covalently bonded to a carbon that is part of a phenyl ring. The term “phenyl ring” should be broadly understood to mean any ring comprising six carbon atoms having three conjugated double bonds. Thus, a phenylsulfonyl moiety could be monosubstituted, meaning that the sulfonyl group is the only group directly attached to the phenyl ring, or the phenylsulfonyl moiety could have from 1 to 5 additional substituents which are not a hydrogen atom, and are directly attached to a carbon of the phenyl ring. In certain embodiments, the prodrug comprises both a phenylsulfonyl moiety and a carboxylic acid or a pharmaceutically acceptable salt thereof.

The term “N-phenylsulfonyl prodrug of a proton pump inhibitor” refers to a prodrug of a proton pump inhibitor having a phenylsulfonyl moiety bonded to a proton pump inhibitor, where said bond occurs between the sulfur atom of the phenylsulfonyl moiety and the nitrogen atom of the proton pump inhibitor.

In certain embodiments the proton pump inhibitor is coupled to an ionic functional group or a conjugate acid or base thereof via a sulfonamide linkage. A sulfonamide linkage comprises a covalent bond between a nitrogen atom and the sulfur atom of an SO₂ moiety, where the sulfur atom is also connected to the ionic functional group or conjugate acid or base thereof via any group of atoms, bonds, or moieties. Thus, while not intending to limit the scope of the invention in any way, the SO₂ group may be directly bonded to the ionic functional group or conjugate acid or base thereof, or the two may be connected by groups such as alkyl, alkenyl, alkynyl, phenyl, napthyl, pyridinyl, alkyloxy, alkenyloxy, alkynyloxy, phenoxy, and the like. In one embodiment, the sulfonamide linkage relates to a phenylsulfonamide, meaning that a phenyl moiety is directly bonded to the SO₂ group. However, in relation to this embodiment, the phenyl group may be directly bonded to the ionic moiety or conjugate acid or base thereof, or the two may be connected indirectly by groups similar to those mentioned above. Additionally, the phenyl ring may have additional substituents, up to the point where every carbon of the ring has a non-hydrogen substituent.

A “sulfonamide of a proton pump inhibitor” as used herein refers to a derivative of a proton pump inhibitor having a direct covalent bond between a nitrogen atom on the proton pump inhibitor and the sulfur atom of a moiety bearing an SO₂ group. In this case, the sulfur atom is bonded directly to two oxygen atoms and the remainder of said moiety, as well as to the nitrogen of the proton pump inhibitor.

The term “solubilizing moiety” as used herein has the broadest meaning generally accepted in the art with reference to a moiety which increases the water solubility of a compound related to a proton pump inhibitor, but which is not present on the proton pump inhibitor. However, a solubilizing moiety may be a duplicate of a moiety present on the parent proton pump inhibitor. Thus, if a proton pump inhibitor has a group X, a second X comprised by a prodrug or other compound related to a proton pump inhibitor would still be considered a solubilizing moiety if X increases the water solubility of the molecule.

Solubilizing moieties are well known by those of ordinary skill in the art. While not intending to limit the scope of the invention in any way, a solubilizing moiety may have one or more of the following features: 1) an ionic charge, 2) a large dipole moment, 3) one or more hydrogen bond donors, and 4) one or more hydrogen bond acceptors. Thus useful solubilizing moieties will include, but are not limited to, moieties comprising an effective amount of hydroxyl functional groups to increase the water solubility, such as sugar-based groups comprising monosaccharide, disaccharide, oligosaccharide, or polysaccharide derivatives, or cyclodextrins and related moieties; polyalkylene oxide groups; and glycerine-based groups. Other useful solubilizing groups comprise ionic functional groups or conjugate acids or bases thereof. An ionic functional group has the broadest meaning generally understood in the art, and refers to a group carrying an ionic charge. A conjugate acid or base of an ionic functional group has the meaning normally understood in the art, i.e. a neutral functional group which is formed by either removing or adding a proton to the ionic functional group. Other useful solubilizing groups comprise an acidic functional group or a pharmaceutically acceptable salt thereof. An “acidic functional group” is defined herein as a functional group with a pKₐ below 10. While not intending to limit the scope of the invention, certain examples of acidic functional groups and/or conjugate acids of ionic functional groups include, but are not limited to, carboxylic acids, sulfonic acids, sulfate esters, phosphonic acids, and phosphate esters. Carboxylic acids and their pharmaceutically acceptable salts are of particular interest as solubilizing moieties related to the compounds disclosed herein.

It is generally understood in the art that the particular form of an ionic, or an acidic or basic functional group, is often dependent upon the pH of the environment around said group. Thus, in relation to certain claim elements, it is understood that the form of certain solubilizing moieties may depend upon pH. Thus, at a low pH, the solubilizing moiety may be in a neutral, conjugate acid, or acidic form. Alternatively, at a high pH, the solubilizing moiety may be in an ionic, basic, or conjugate base form. Thus, in cases where it is not expressly stated otherwise, these considerations are implicit in the proper interpretation of the relevant claim elements. For example, while not intending to limit the scope of the invention in any way, in an aqueous liquid of pH 7.4 comprising a carboxylic acid with a pKₐ of 3, it is understood that the carboxylic acid will be predominantly in the ionic form, unless some unusual circumstance affects the properties of the acid. In the case of solid compositions, it is understood that the form of the solubilizing moiety may vary according to a number of factors which are related to acid-base reactions, pH considerations of certain claim elements, and the methods in which said compositions are prepared.

A “pharmaceutically acceptable salt” is any salt that retains the activity of the parent compound and does not impart any deleterious or untoward effect on the subject to which it is administered and in the context in which it is administered as compared to the parent compound.

Pharmaceutically acceptable salts of acidic functional groups may be derived from organic or inorganic bases. The salt may be a mono or polyvalent ion. Of particular interest are the inorganic ions lithium, sodium, potassium, calcium, and magnesium. Organic salts may be made with amines, particularly ammonium salts such as mono-, di- and trialkyl amines or ethanol amines. Salts may also be formed with caffeine, tromethamine and similar molecules. Hydrochloric acid or some other pharmaceutically acceptable acid may form a salt with a compound that includes a basic group, such as an amine or a pyridine ring.

While not intending to limit the scope of the invention in any way, in many situations one practicing the invention might choose a compound which would be converted after administration into one of the widely used and well tested commercially available proton pump inhibitors (PPI) such as lansoprazole, esomeprazole, omeprazole, pantoprazole, and rabeprazole. In situations where one of the commercially available PPIs is used as the PPI in one of the embodiments, an artisan may want to consider circumstances related to the individual to which the prodrug is administered in making decisions related to features of a particular embodiment. For example, if the person to which the prodrug is being administered is known to respond well to omeprazole, then one may consider practicing an embodiment comprising a prodrug of omeprazole. In another situation, a person may have a history of being effectively treated by lansoprazole, in which case one may consider practicing an embodiment comprising a prodrug of lansoprazole. The specific examples related to proton pump inhibitor are given merely to provide guidance and direction to one practicing one or more of embodiments disclosed herein, and are not intended to limit the overall scope of the invention in any way. In one embodiment the proton pump inhibitor is lansoprazole. In another embodiment the proton pump inhibitor is omeprazole. In another embodiment the proton pump inhibitor is esomeprazole. In another embodiment the proton pump inhibitor is pantoprazole. In another embodiment the proton pump inhibitor is rabeprazole.

Certain embodiments relate to particular structures, which are useful as prodrugs.
One embodiment comprises 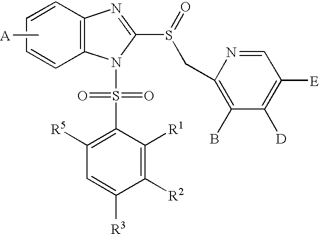
or a pharmaceutically acceptable salt thereof;
wherein
A is H, OCH₃, or OCHF₂;
B is CH₃ or OCH₃;
D is OCH₃, OCH₂CF₃, or O(CH₂)₃OCH₃;
E is H or CH₃;
R¹, R², R³, and R⁵ are independently H, CH₃, CO₂H, CH₂CO₂H, (CH₂)₂CO₂H, CH(CH₃)₂, OCH₂C(CH₃)₂CO₂H, OCH₂CO₂CH₃, OCH₂CO₂H, OCH₂CO₂NH₂, OCH₂CONH₂(CH₂)₅CO₂CH₃, or OCH₃, provided that at least one of R¹, R², R³, and R⁵ comprises a carboxylic acid functional group.

In another embodiment related to the one just described, R¹, R², R³, and R⁵ are independently H, CH₃, CO₂H, CH₂CO₂H, (CH₂)₂CO₂H, OCH₂CO₂CH₃, OCH₂CO₂H, OCH₂CONH₂(CH₂)₅CO₂CH₃, or OCH₃.

In certain embodiments, the prodrug has a structure comprising

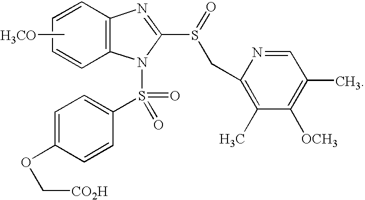

In other embodiments, the prodrug has a structure comprising

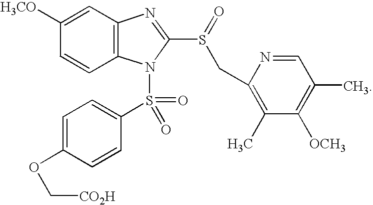

In other embodiments, the prodrug has a structure comprising

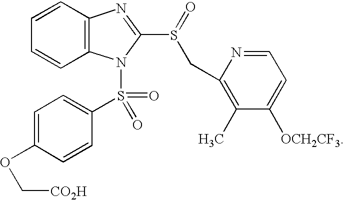

In other embodiments, the prodrug has a structure comprising

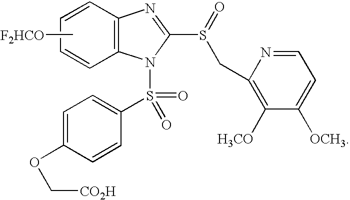

In other embodiments, the prodrug has a structure comprising

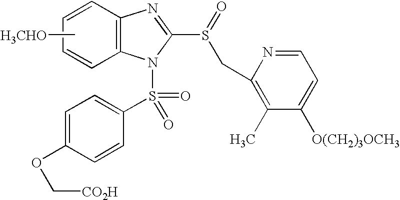

The prodrugs of the present invention can be prepared by the methods described in the following U.S. Patent documents, all of which are expressly incorporated by reference herein: U.S. Pat. No. 6,093,734; U.S. patent application Ser. No. 09/783,807, filed Feb. 14, 2001; the U.S. Pat. App. having the title “PRODRUGS OF PROTON PUMP INHIBITORS”, filed Jul. 15, 2003 by applicants Michael E. Garst, George Sachs, and Jai M. Shin, which has not yet been assigned a serial number; and the U.S. Pat. App. having the title “PROCESS FOR PREPARING ISOMERICALLY PURE PRODRUGS OF PROTON PUMP INHIBITORS”, filed Jul. 15, 2003 by applicants Michael E. Garst, Lloyd J. Dolby, Shervin Esfandiari, Vivian R. Mackenzie, Alfred A. Avey, Jr., David C. Muchmore, Geoffrey K. Cooper, and Thomas C. Malone, which has not yet been assigned a serial number. However, these methods are only given to provide guidance, and are not meant to limit the scope of the invention in any way. One of ordinary skill in the art will recognize that there are many ways in which the prodrugs of the present invention can be prepared without departing from the spirit and scope of the present invention.

Parenteral administration is generally characterized by injection. Injectables can be prepared in conventional forms, either as liquid solutions or suspensions, solid forms suitable for dissolving or suspending in liquid prior to injection, or as emulsions. Descriptions of substances and methods normally used to prepare formulations for parenteral administration can be found in several treatises and books well known in the art such as, Handbook On Injectable Drugs (11th edition), edited by Lawrence A. Trissel, (Chicago: Login Brothers Book Company; Jan. 15, 2001).

The term “reconstituted” refers broadly to any process where a compound disclosed herein becomes dissolved in water or any aqueous solution. While not intending to limit the scope of the invention in any way, for example, the solid may be directly dissolved in water or in an aqueous solution. Alternatively, the solid may be dissolved in water or another aqueous solution, then further diluted one or more times by water or another aqueous solution. While not intending to limit the scope of the invention in any way, the aqueous solution referred to may comprise any compound which is compatible with the use of the solid, such as a tonicity agent, a sugar, or a buffer.

The following examples provide guidance and direction in making and using the invention, and demonstrate the advantages of the present invention, and are not to be interpreted as limiting the scope of the invention in any way.

### EXAMPLE 1

Compounds specifically contemplated in relation to embodiments disclosed herein are presented in Table 1 below. The generic structure, I, is shown as a combination of a proton pump inhibitor (X) and a sulfonyl-bearing moiety which is attached to the proton pump inhibitor to form the prodrug according to the formula below. The identity of each group represented by R¹-R⁵ is shown in the table.

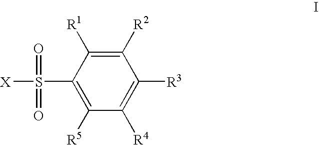

The different possibilities for X are shown below.

TABLE 1

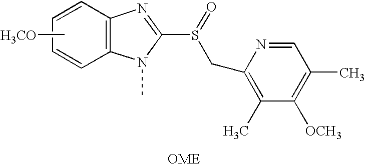

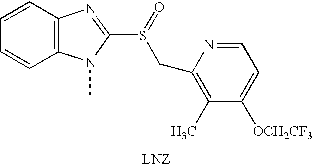

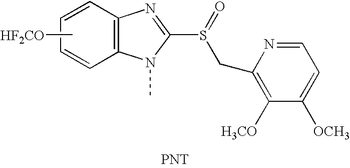

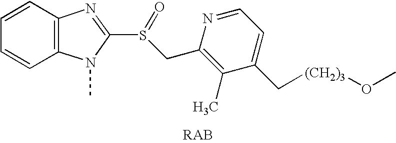

| Compound | X | R¹ | R² | R³ | R⁴ | R⁵ |
|---|---|---|---|---|---|---|
| 1 | OME | H | H | OCH₂CO₂H | H | H |
| 2 | OME | CH₃ | H | OCH₂CO₂H | H | CH₃ |
| 3 | OME | H | H | OCH₂C(CH₃)₂CO₂H | H | H |
| 4 | OME | CH₃ | H | OCH₂C(CH₃)₂CO₂H | H | CH₃ |
| 5 | OME | H | H | CH₂CO₂H | H | H |
| 6 | OME | H | CO₂H | H | H | H |
| 7 | LNZ | H | CO₂H | H | H | H |
| 8 | LNZ | H | CO₂H | OCH₃ | H | H |
| 9 | LNZ | H | H | CH₂CO₂H | H | H |
| 10 | LNZ | H | H | OCH₂CO₂H | H | H |
| 11 | LNZ | H | H | OCH₂C(CH₃)₂CO₂H | H | H |
| 12 | LNZ | H | CH₂CO₂H | CH₂CO₂H | H | H |
| 13 | LNZ | H | CO₂H | H | H | CH₃ |
| 14 | LNZ | H | CO₂H | H | H | OCH₃ |
| 15 | LNZ | CH(CH₃)₂ | H | CH₂CO₂H | H | H |
| 16 | LNZ | H | OCH₂CO₂H | CO₂H | H | H |
| 17 | LNZ | CH(CH₃)₂ | H | OCH₂CO₂H | H | CH₃ |
| 18 | LNZ | H | H | CO₂H | H | H |
| 19 | LNZ | H | (CH₂)₂CO₂H | CH₃ | H | H |
| 20 | OME | H | H | OCH₂CO₂CH₃ | H | H |
| 21 | OME | H | H | OCH₂CO₂NH₂ | H | H |
| 22 | OME | H | CO₂H | CO₂H | H | H |
| 23 | OME | H | CO₂H | OCH₂CO₂H | H | H |
| 24 | OME | H | OCH₂CO₂H | OCH₂CO₂H | H | H |
| 25 | OME | OCH₃ | H | CO₂H | H | H |
| 26 | OME | H | | CO₂H | H | H |
| 27 | OME | H | CO₂H | H | H | CH₃ |
| 28 | PNT | H | H | OCH₂CO₂H | H | H |
| 29 | PNT | H | CO₂H | H | H | CH₃ |
| 30 | RAB | H | CO₂H | H | H | H |
| 31 | RAB | H | CO₂H | H | H | CH₃ |
| 32 | RAB | CH₃ | H | OCH₂CO₂H | H | CH₃ |
| 33 | RAB | H | H | CO₂H | H | H |
| 34 | LNZ | CH₃ | H | OCH₂CO₂H | H | CH₃ |
| 35 | LNZ | H | OCH₂CO₂H | OCH₂CO₂H | H | H |
| 36 | LNZ | H | H | CO₂H | H | H |
| 37 | LNZ | CH₃ | H | CO₂H | H | H |
| 38 | LNZ | H | (CH₂)₂CO₂H | OCH₃ | H | H |
| 39 | OME | CH₃ | H | OCH₂CONH₂(CH₂)₅ | H | CH₃ |
| | | | | CO₂CH₃ | | |
| 40 | OME | H | H | OCH₂CONH₂(CH₂)₅ | H | H |
| | | | | CO₂CH₃ | | |
| 41 | OME | H | H | (CH₂)₂CO₂H | H | H |
| 42 | OME | H | (CH₂)₂CO₂H | OCH₃ | H | H |

These compounds have been prepared according to procedures described the U.S. Pat. App. having the title “PRODRUGS OF PROTON PUMP INHIBITORS”, filed Jul. 15, 2003 by applicants Michael E. Garst, George Sachs, and Jai M. Shin, which has not yet been assigned a serial number; and the U.S. Pat. App. having the title “PROCESS FOR PREPARING ISOMERICALLY PURE PRODRUGS OF PROTON PUMP INHIBITORS”, filed Jul. 15, 2003 by applicants Michael E. Garst, Lloyd J. Dolby, Shervin Esfandiari, Vivian R. Mackenzie, Alfred A. Avey, Jr., David C. Muchmore, Geoffrey K. Cooper, and Thomas C. Malone, which has not yet been assigned a serial number, incorporated by reference previously herein. These aforementioned patent documents, as well as the provisional U.S. Patent Application having the title “METHODS AND COMPOSITIONS FOR THE ORAL ADMINISTRATION OF PRODRUGS OF PROTON PUMP INHIBITORS”, which was filed on Oct. 3, 2003 by applicants Jie Shen, Devin F. Welty, and Diane D. Tang-Liu, incorporated herein by reference, demonstrate that compounds 1-42 decompose in vivo to form proton pump inhibitors.

### EXAMPLE 2

The physicochemical properties of compound 1 were analyzed. Compound 1 was found to be hygroscopic, in that 9% weight gain was observed for the compound after 14 days of storage at 25° C. at 75% relative humidity.

TABLE 2A
Solubility Profile of Compound 1
at 25° C. in Buffered Aqueous Solutions

| | | Solubility |
|---|---|---|
| pH | Buffer Composition | (mg/mL) |
| 1 | 0.1 M HCl | 1.8 |
| 3 | Citric Acid (0.1 M)/ | 0.4 |
| | Na₂HPO₄ (0.2 M) | |
| 5 | Citric Acid (0.1 M)/ | >50 |
| | Na₂HPO₄ (0.2 M) | |
| 7 | sodium phosphate (0.1-0.2 M) | >50 |
| 9 | sodium phosphate (0.1-0.2 M) | >50 |

The solubility profile of compound 1 in at various pH values is presented in Table 2A. This data shows that the aqueous solubility of the compound is significantly enhanced at around pH 5. While not intending to be bound in any way by theory, it is believed that this improvement in solubility is due to the deprotonation of a sufficient quantity of the acid. While not intending to be bound in any way by theory, this suggests that the compounds comprising a carboxylic acid should be significantly easier to formulate in the compositions and dosage forms disclosed herein.

TABLE 2A
Stability Profile of Compound 1 at 25° C. in Buffered
Aqueous Solutions

| | | Half-life | | Degradation |
|---|---|---|---|---|
| | Buffer | (t_{1/2}) | Shelf life | Rate Constant (k) |
| pH | Composition | hours | (t_{90%}) hours | 1/hours |
| 1 | 0.1 M HCl | 3.6 | 0.5 | 0.194 |
| 3 | Citric Acid (0.1 M)/ | 78.0 | 11.9 | 0.009 |
| | Na₂HPO₄ (0.2 M) | | | |
| 5 | Citric Acid (0.1 M)/ | 89.2 | 13.6 | 0.008 |
| | Na₂HPO₄ (0.2 M) | | | |
| 7 | sodium phosphate | 286.8 | 43.6 | 0.002 |
| | (0.1-0.2 M) | | | |
| 7.4 | sodium phosphate | 291.2 | 44.3 | 0.002 |
| | (0.1-0.2 M) | | | |
| 9 | sodium phosphate | 23.0 | 3.5 | 0.030 |
| | (0.1-0.2 M) | | | |
| 10 | sodium phosphate | 2.3 | 0.4 | 0.298 |
| | (0.1-0.2 M) | | | |

The aqueous stability data of compound 1 is presented in Table 2B. While not intending to be bound in any way by theory, the base-catalyzed degradation is unexpected because the commercial proton pump inhibitors are stabilized in aqueous solutions by adjusting the solution to high pH. In fact, while not intending to be bound or limited in any way by theory, compound 1 appears to be more susceptible to base-catalyzed degradation than acid-catalyzed degradation, since its half-life is longer at pH 5, where the H⁺ concentration is 10⁻⁵ M than its half-life is at pH 9, where the OH⁻ concentration is 10⁻⁵ M. Similarly, compound 1 is less stable at pH 10, where the OH⁻ concentration is 10⁻⁴ M than it is at pH 1, where the H⁺ concentration is 0.1 M. While not intending to be bound or limited in any way by theory, these results unexpectedly show that, in contrast to the proton pump inhibitors, these compounds are more stable in neutral and acidic solutions than they are at a higher pH. In the proper pH range, they are also significantly more stable than the current commercial proton pump inhibitors. In a neutral solution, the shelf-life of compound 1 is over forty hours. As low as around pH 3, compound 1 has a sufficient shelf life to be comparable to the commercial omeprazole intravenous formulation Losec®, which is reported by the manufacturer, Astra Zeneca, to have a shelf life of “12 hours when dissolved in normal saline and 3 hours in 5% dextrose when stored at 25° C.” [http://www.astrazeneca.co.uk/downloads/LosecInfusion(9476).pdf]. Thus, the prodrugs disclosed herein are both more stable and more flexible in terms of their use in acidic and neutral aqueous solutions as compared to the commercial proton pump inhibitor products currently available. This should allow bolus injection of the compounds disclosed herein as opposed to the slow infusion of the drug currently in practice because the present compositions will not have the irritation associated with the high pH traditionally used with proton pump inhibitors. This should also allow greater flexibility in co-administering these compounds with drugs which are unstable or otherwise incompatible with high pH. Additionally, while not intending to be bound in any way by theory, or to limit the invention in any way, these results also demonstrate that the solid will be easier to handle, because moisture is less likely to destabilize the active compound.

### EXAMPLE 3

Compound 1 (125 mg) is dissolved in 100 mL of water, and the solution is administered intravenously to a person to inhibit gastric acid secretion in that person.

What is claimed is:

1. A composition comprising a therapeutically effective concentration of an N-phenylsulfonyl prodrug of a proton pump inhibitor comprising a solubilizing moiety, wherein said composition is an aqueous liquid having a pH of from 3 to 7.3.

2. The composition of claim 1 wherein said solubilizing moiety comprises an acidic functional group or a pharmaceutically acceptable salt thereof.

3. The composition of claim 1 wherein said solubilizing moiety comprises one or more hydroxyl functional groups.

4. The composition of claim 1 wherein said solubilizing moiety comprises a carboxylic acid or a pharmaceutically acceptable salt thereof.

5. The composition of claim 1 wherein the pH is from 5 to 7.

6. The composition of claim 1 wherein the pH is from 5 to 6.

7. The composition of claim 1 wherein the pH is about 5.5.

8. The composition of claim 1 comprising 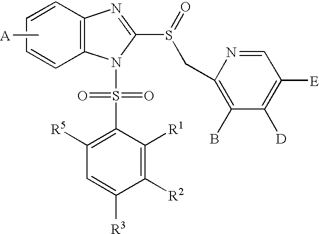
or a pharmaceutically acceptable salt thereof;
wherein
A is H, OCH₃, or OCHF₂;
B is CH₃ or OCH₃;
D is OCH₃, OCH₂CF₃, or O(CH₂)₃OCH₃;
E is H or CH₃;
R¹, R², R³, and R⁵ are independently H, CH₃, CO₂H, CH₂CO₂H, (CH₂)₂CO₂H, CH(CH₃)₂, OCH₂C(CH₃)₂CO₂H, OCH₂CO₂CH₃, OCH₂CO₂H, OCH₂CO₂NH₂, OCH₂CONH₂(CH₂)₅CO₂CH₃, or OCH₃, provided that at least one of R¹, R², R³, and R⁵ comprises a carboxylic acid functional group.

9. The composition of claim 8 wherein R¹, R², R³, and R⁵ are independently H, CH₃, CO₂H, CH₂CO₂H, (CH₂)₂CO₂H, OCH₂CO₂CH₃, OCH₂CO₂H, OCH₂CONH₂(CH₂)₅CO₂CH₃, or OCH₃.

10. The composition of claim 1 wherein the prodrug has a structure comprising

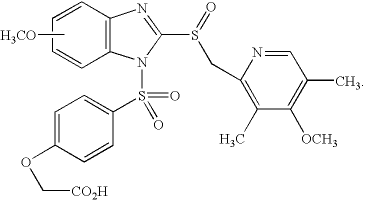

11. The composition of claim 1 wherein the prodrug has a structure comprising

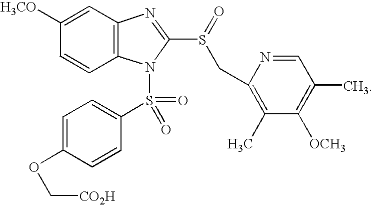

12. The composition of claim 1 wherein the prodrug has a structure comprising

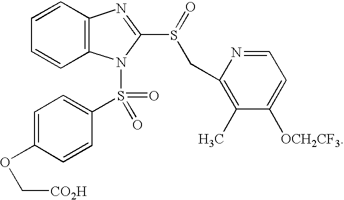

13. The composition of claim 1 wherein the prodrug has a structure comprising

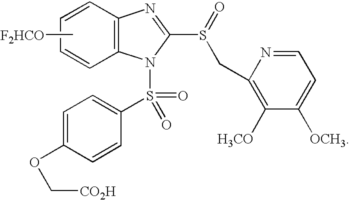

14. The composition of claim 1 wherein the prodrug has a structure comprising

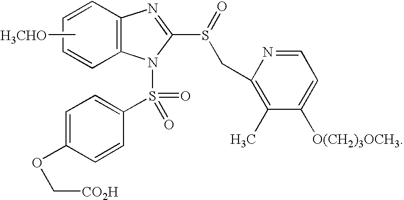

15. A solid composition comprising a prodrug of a proton pump inhibitor comprising a sulfonyl moiety and a carboxylic acid or a pharmaceutically acceptable salt thereof, said solid composition having a pH which is greater than 3 and less than or equal to 7 when dissolved in water at a therapeutically effective concentration for intravenous administration of said prodrug.

16. The composition of claim 15 wherein said proton pump inhibitor is selected from the group consisting of omeprazole, lansoprazole, rabeprazole, pantoprazole, and esomeprazole.

17. The composition of claim 15 wherein said prodrug comprises a phenylsulfonyl moiety.

18. The composition of claim 15 wherein the pH is greater than 3 and less than or equal to 6.

19. The composition of claim 15 wherein the pH is from 6 to 7.

20. The composition of claim 15 wherein the pH is about 6.

21. The composition of claim 20 wherein the prodrug has a structure comprising

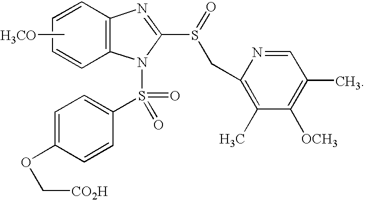

22. The composition of claim 20 wherein the prodrug has a structure comprising

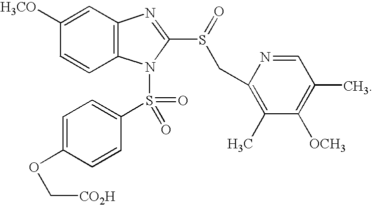

23. A method of delivering a proton pump inhibitor to a mammal comprising
a. dissolving in an aqueous solution a therapeutically effective amount of a proton pump inhibitor which is coupled to an ionic functional group or a conjugate acid or base thereof via a sulfonamide linkage; and
b. administering said aqueous solution parenterally to said mammal;
wherein said aqueous solution has a pH which is greater than or equal to 3 and less than 7.

24. The method of claim 23 wherein said ionic functional group or said conjugate acid or base thereof comprises a carboxylic acid or a pharmaceutically acceptable salt thereof.

25. The method of claim 23 wherein said sulfonamide linkage relates to a phenylsulfonamide.

26. The method of claim 23 wherein said proton pump inhibitor comprises omeprazole.

27. The method of claim 23 wherein said proton pump inhibitor comprises lansoprazole.

28. The method of claim 23 wherein said proton pump inhibitor comprises rabeprazole.

29. The method of claim 23 wherein said proton pump inhibitor comprises pantoprazole.

30. The method of claim 23 wherein said proton pump inhibitor comprises esomeprazole.

31. The method of claim 23 wherein the pH is greater than or equal to 4 and less than 7.

32. The method of claim 23 wherein the pH is from 3 to 4.5.

33. The method of claim 23 wherein the pH is greater than or equal to about 5.5 and less than 7.

34. The method of claim 23 wherein the prodrug has a structure comprising

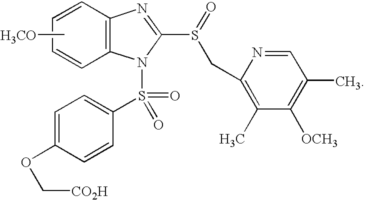

35. The method of claim 23 wherein the prodrug has a structure comprising

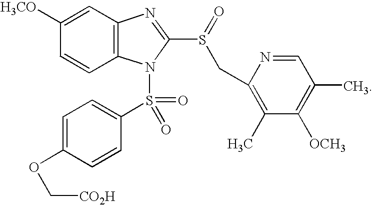

36. A liquid composition comprising a sulfonamide of a proton pump inhibitor and a second therapeutically active agent, said composition having a pH of from 3 to 8.

37. A solid composition comprising a sulfonamide of a proton pump inhibitor and a second therapeutically active agent, said composition having a pH of from 3 to 8 when said composition is dissolved in water at a concentration that is therapeutically effective for parenteral administration of the sulfonamide of a proton pump inhibitor.
